(19) United States
(12) Patent Application Publication
(10) Pub. No.: US 2026/0057512 A1
(43) Pub. Date: Feb. 26, 2026

(54) INFORMATION PROCESSING SYSTEM, INFORMATION PROCESSING METHOD, COMPUTER READABLE STORAGE MEDIUM, AND CELL MANUFACTURING METHOD

(71) Applicants: CANON KABUSHIKI KAISHA, Tokyo (JP); CANON MEDICAL SYSTEMS CORPORATION, Tochigi (JP)

(72) Inventors: MIE OKANO, Kanagawa (JP); YUKITERU MASUDA, Kanagawa (JP); YOJI YAMAMOTO, Tokyo (JP); SHUHEI TOBA, Kanagawa (JP)

(21) Appl. No.: 19/306,808

(22) Filed: Aug. 21, 2025

(30) Foreign Application Priority Data
Aug. 23, 2024 (JP) 2024-143346
Aug. 20, 2025 (JP) 2025-137192

(51) Int. Cl. G06T 7/00; C12M 1/34; C12N 5/074; G06V 20/69

(52) U.S. Cl. CPC G06T 7/0012; C12M 41/46; C12N 5/0696; G06V 20/698; G06T 2207/20081; G06T 2207/30024; G06T 2207/30242

(57) ABSTRACT

Tests regarding chromosomal abnormalities in pluripotent stem cells have involved cellular invasion, and have been unable to be applied to cells that require non-invasive testing, such as cells being cultured. Provided is an information processing system including: an image data acquisition unit configured to acquire image data including a colony of pluripotent stem cells; a first information acquisition unit configured to acquire, based on analysis of the image data, first information that is information regarding a predetermined morphology of the colony of pluripotent stem cells; and a second information generation unit configured to generate, based on the first information, second information that is information regarding chromosomal abnormalities in the pluripotent stem cells.

### BACKGROUND

### Field of the Technology

The present disclosure relates to an information processing system, an information processing method, a computer readable storage medium, and a cell manufacturing method that enable acquisition of information regarding chromosomal abnormalities in pluripotent stem cells.

### Description of the Related Art

Of pluripotent stem cells, induced pluripotent stem cells, known as iPS cells, have unlimited proliferative ability and pluripotency, and are thus anticipated to be put into practical use in fields such as regenerative medicine and drug discovery research. However, it is known that karyotype abnormalities occur when pluripotent stem cells are cultured for a long period of time. The karyotype abnormalities frequently occur, and hence chromosomal abnormality testing (G-band analysis) has become an essential test item in quality control of iPS cells.

However, the G-band analysis is a destructive test, and also requires a great deal of time, effort, and cost. In contrast, the chromosomal abnormalities may randomly occur in cells being cultured, and regular testing of pluripotent stem cells being cultured is required. For those reasons, there is a demand for a non-destructive, rapid, and highly sensitive technology that enables detection of cells with chromosomal abnormalities that occur during a culture process.

Shapes of colonies and cells are important for evaluating states of iPS cells, and hence there are a large number of researches being conducted, such as a research into evaluation of quality of iPS cells to be performed by a test method using image information and a research into automatic detection of chromosomal abnormalities in iPS cells to be performed in a non-destructive manner through use of image information and an artificial intelligence (AI). However, in those research, single cells are analyzed, and it is required to introduce fluorescent dyes into cells or pick up images showing chromosomes themselves, thereby disabling non-invasive detection during culture. Further, a morphology of a colony is not used for non-invasive testing for karyotype abnormalities.

In Japanese Patent Laid-Open No. 2022-551683, presence of chromosomal abnormalities is non-invasively estimated by an AI-based computer calculation system through use of images of embryos prior to implantation. However, this technology is directed to embryos, and it is not specifically described what kind of characteristics of the embryo images is used to estimate the presence of chromosomal abnormalities.

In Japanese Patent Laid-Open No. 2024-510103, image data on cells is processed to classify the cells through use of morphological characteristics of the cells. However, this technology is directed to single cells, and morphological characteristics specific to colonies are not used for the classification.

### SUMMARY

According to one embodiment of the present disclosure, there is provided an information processing system including: an image data acquisition unit configured to acquire image data including a colony of pluripotent stem cells; a first information acquisition unit configured to acquire, based on analysis of the image data, first information that is information regarding a predetermined morphology of the colony of pluripotent stem cells; and a second information generation unit configured to generate, based on the first information, second information that is information regarding chromosomal abnormalities in the pluripotent stem cells.

Further, according to another embodiment of the present disclosure, there is provided an information processing method including: acquiring image data including a colony of pluripotent stem cells; acquiring, based on analysis of the image data, first information regarding a predetermined morphology of the colony of pluripotent stem cells; and generating, based on the first information, second information that is information regarding chromosomal abnormalities in the pluripotent stem cells.

Further, according to still another embodiment of the present disclosure, there is provided a cell manufacturing method including: acquiring image data including a colony of pluripotent stem cells; acquiring, based on analysis of the image data, first information that is information regarding a predetermined morphology of the colony of pluripotent stem cells; generating, based on the first information, second information that is information regarding chromosomal abnormalities in the pluripotent stem cells; and classifying, based on the second information, pluripotent stem cells being cultured which include the pluripotent stem cells.

Features of the present disclosure will become apparent from the following description of embodiments with reference to the attached drawings. The following description of embodiments is described by way of example.

### BRIEF DESCRIPTION OF THE DRAWINGS

FIG. 1 is a flow chart for illustrating an example of a flow in a case of using an information processing apparatus according to this embodiment.

FIG. 2 is a diagram for illustrating an example of a configuration of the information processing apparatus according to this embodiment.

FIG. 3 is a diagram for illustrating an example of a configuration of the information processing apparatus according to this embodiment.

FIG. 4A shows an example of a mask image of a colony before analysis.

FIG. 4B shows an example of spikes of a colony after analysis.

FIG. 4C shows an example of a particle image created from the mask image of the colony before analysis.

FIG. 5 shows a cell major axis that exemplifies information regarding a cell present in an outer peripheral portion of a colony.

FIG. 6A is a conceptual diagram for illustrating an example of a flow in a case of using a trained model.

FIG. 6B is a conceptual diagram for illustrating an example of a flow in a case of using the trained model.

FIG. 6C is a conceptual diagram for illustrating an example of a flow in a case of using a corresponding relationship between first information and second information.

FIG. 6D is a conceptual diagram for illustrating an example of a flow in a case of using a corresponding relationship between the first information, the second information, and third information.

FIG. 6E is a conceptual diagram for illustrating an example of a flow in a case of using a first trained model and a second trained model.

FIG. 7 is a view for illustrating an example of the first information and the third information acquired in advance.

FIG. 8A is a view for illustrating an example of display in a case of outputting the second information.

FIG. 8B is a view for illustrating an example of display in the case of outputting the second information.

FIG. 8C is a view for illustrating an example of display in the case of outputting the second information.

FIG. 8D is a view for illustrating an example of display in the case of outputting the second information.

FIG. 9 is a flow chart for illustrating an information processing flow for pluripotent stem cells in a case of an inconclusive determination in this embodiment.

FIG. 10A is a view for illustrating an example of display in the case of an inconclusive determination.

FIG. 10B is a view for illustrating an example of display in the case of an inconclusive determination.

FIG. 10C is a view for illustrating an example of display in the case of an inconclusive determination.

FIG. 10D is a view for illustrating an example of display in the case of an inconclusive determination.

FIG. 11 is a flow chart for illustrating an information processing flow for pluripotent stem cells in the case of an inconclusive determination.

FIG. 12 is a view for illustrating an example of display of determination results based on a plurality of colonies.

FIG. 13 is an example of a flow of manufacturing pluripotent stem cells.

FIG. 14 is a view for illustrating an example of data acquired in this embodiment.

FIG. 15 is a view for illustrating data regarding the number of spikes acquired in advance and stored in the information processing apparatus in Example 1.

FIG. 16 is a view for illustrating an example of display of the second information regarding chromosomal abnormalities acquired in Example 2.

FIG. 17 is a graph for showing data on sizes of nuclei of cells in the outer peripheral portion which are acquired in advance and stored in the information processing apparatus in Example 3.

### DESCRIPTION OF THE EMBODIMENTS

An embodiment is described in detail below with reference to the attached drawings. It should be noted that the present disclosure is specified by the claims, and is not construed as being limited to the following modes.

### <Information Processing Apparatus>

An information processing apparatus according to one embodiment of the present disclosure includes an image data acquisition unit that acquires image data including a colony of pluripotent stem cells, a first information acquisition unit that acquires first information, which is information regarding a predetermined morphology of the colony of pluripotent stem cells, based on analysis of the image data, and a second information generation unit that generates second information, which is information regarding chromosomal abnormalities in the pluripotent stem cells, based on the first information. The information processing apparatus according to one embodiment of the present disclosure may be hereinafter referred to simply as “information processing apparatus according to the present disclosure.”

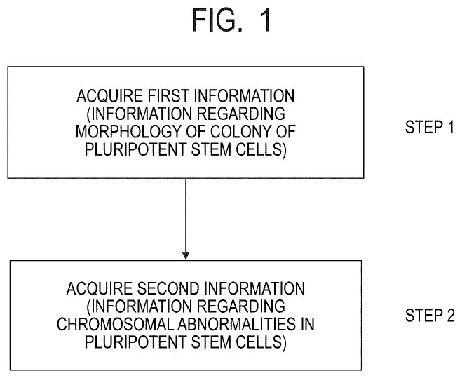

FIG. 1 is a flow chart for illustrating a flow in a case of using the information processing apparatus according to the present disclosure to acquire the information regarding the chromosomal abnormalities. In the present disclosure, the image data including the colony of pluripotent stem cells is acquired as Step 1. After that, the second information, which is the information regarding the chromosomal abnormalities in the pluripotent stem cells, is acquired as Step 2 based on first information, which is the information regarding the predetermined morphology of the colony, and has been obtained by analyzing the image data on an image including the colony.

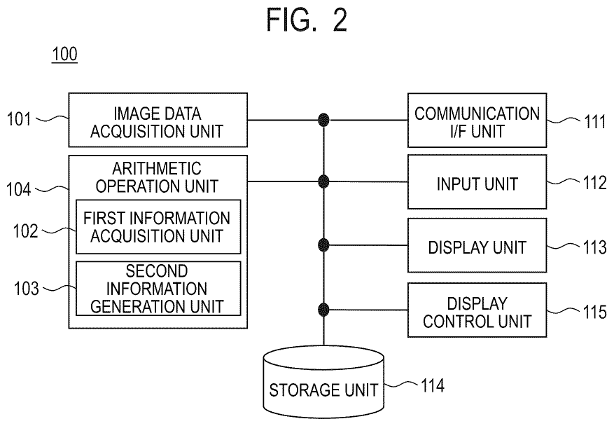

In FIG. 2, an example of a configuration of an information processing apparatus 100 according to the present disclosure is illustrated. The information processing apparatus 100 has functions of a computer. The information processing apparatus 100 according to the present disclosure is only required to include an image data acquisition unit 101, and a first information acquisition unit 102 and a second information generation unit 103 that are included in an arithmetic operation unit 104, and other units are not essential.

For example, the information processing apparatus 100 may be a desktop personal computer (PC), a laptop PC, a tablet PC, or a smartphone.

The information processing apparatus 100 that performs arithmetic operations and recording can include a storage unit 114, the image data acquisition unit 101, the arithmetic operation unit 104 having functions of the first information acquisition unit 102 and the second information generation unit 103, a communication interface (I/F) unit 111, an input unit 112, a display unit 113, and a display control unit 115 in order to implement functions of a computer that performs the arithmetic operations and the recording. In FIG. 2, the respective units that form the information processing apparatus 100 are illustrated as an integrated apparatus, but some of those functions may be externally provided. For example, the display unit 113 and the input unit 112 may be externally provided (may each be present as an apparatus different from the information processing apparatus 100).

The storage unit 114 is, for example, a random access memory (RAM), a read only memory (ROM), or a hard disk drive (HDD). The first information, the second information, and the like can be recorded in the storage unit 114.

The image data acquisition unit 101 acquires image data including the colony of pluripotent stem cells as Step 1 of the flow illustrated in FIG. 1. The image data acquisition unit 101 included in the information processing apparatus 100 may be a photographing unit present in the information processing apparatus 100 such as a microscope or a camera or an image data acquisition unit having a function of acquiring image data through photographing in cooperation with the photographing unit, or may be an image data acquisition unit having a function of acquiring through the communication I/F unit 111 or the storage unit 114, for example, image data stored in a server such as image data acquired by a photographing unit included in an apparatus or the like outside the information processing apparatus 100 or image data acquired through photographing in the past, or image data acquired through photographing by a photographing unit of another apparatus. The image data including the colony of pluripotent stem cells is image data including a colony of unstained pluripotent stem cells, and is preferred to be, for example, image data on an image picked up by phase contrast, differential interference, dark-field, oblique illumination, or transmission defocus. Further, in this embodiment, the information processing apparatus 100 includes the image data acquisition unit 101, but an apparatus other than the information processing apparatus 100 may include the image data acquisition unit 101 and a first information generation unit that generates first information by analyzing image data acquired by the image data acquisition unit 101, or the information processing apparatus 100 according to this embodiment may include the first information generation unit or the first information generation unit and the image data acquisition unit.

The arithmetic operation unit 104 is, for example, a CPU, and has a function of performing a predetermined operation in accordance with a program stored in the RAM, the HDD, or the like, and controlling each unit of the information processing apparatus 100. The arithmetic operation unit 104 further receives data from the storage unit 114, the input unit 112, the communication I/F unit 111, and the like, and performs control and an arithmetic operation on the computer.

The arithmetic operation unit 104 has the functions of the first information acquisition unit 102 that acquires the first information, which is the information regarding the predetermined morphology of the colony of pluripotent stem cells, and has been generated by analyzing the image data including the colony of pluripotent stem cells, and the second information generation unit 103 that generates the second information, which is the information regarding the chromosomal abnormalities in the pluripotent stem cells, based on the acquired first information. The arithmetic operation unit 104 may also have a role of sending the second information generated by the second information generation unit 103 to the storage unit, or the display control unit 115 may perform control to display the first information and the second information on the display unit. In this case, in regard to the analysis of the image data including the colony of pluripotent stem cells, as described later, a trained model may be used to generate and acquire first information, which is the information regarding the predetermined morphology of the colony of pluripotent stem cells, from the image data including the colony of pluripotent stem cells, the trained model having been trained so as to output the first information, which is the information regarding the predetermined morphology of the colony, through use of training data (data set) including images including colonies of pluripotent stem cells having chromosomal abnormalities and images including colonies of pluripotent stem cells having no chromosomal abnormality. The information regarding the “predetermined” morphology of the colony of pluripotent stem cells as used herein refers to information regarding a morphology selected or defined in advance for the colony of pluripotent stem cells.

The communication I/F unit 111 is a communication interface based on standards such as Wi-Fi (trademark), 4G, and 5G, and is a module for communicating to/from another apparatus. For example, image data including colonies of pluripotent stem cells acquired by the photographing unit such as a microscope can be acquired by the image data acquisition unit 101 or can be stored in the storage unit 114, through Wi-Fi.

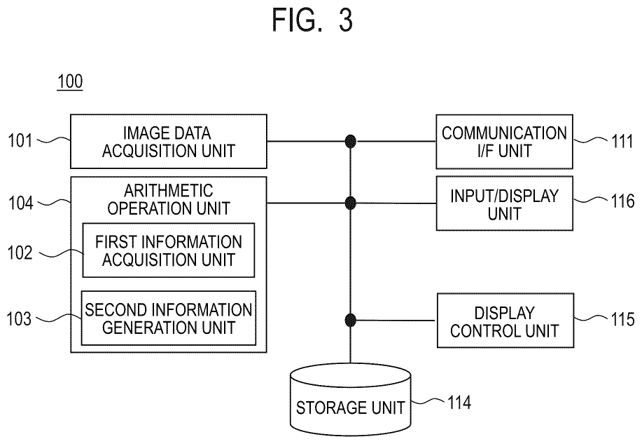

The input unit 112 is a button, a keyboard, a pointing device, or the like, and is used by a user to operate the information processing apparatus 100. The first information, which is the information regarding the predetermined morphology of the colony, and has been acquired by the first information acquisition unit 102, information different from the first information which is acquired in advance, and the like can also be input to the information processing apparatus 100. The display unit 113 and the input unit 112 may be integrally formed as an input/display unit 116 such as a touch panel as illustrated in FIG. 3.

The display unit 113 may be a liquid crystal display, an organic light emitting diode (OLED) display, or the like, and is used to display moving images, still images, characters, and the like. The display unit 113 may display any one of the first information acquired by the first information acquisition unit 102 or the second information generated by the second information generation unit 103, or both thereof.

The information processing apparatus 100 may also include the display control unit 115 for causing the display unit 113 to perform display.

### <Information Processing System>

The present disclosure further provides the following information processing system. An information processing system according to the present disclosure includes an image data acquisition unit that acquires image data including a colony of pluripotent stem cells, a first information acquisition unit that acquires first information, which is information regarding a predetermined morphology of the colony, and a second information generation unit that generates second information, which is information regarding chromosomal abnormalities in the pluripotent stem cells, based on the first information.

In this case, the image data acquisition unit, the first information acquisition unit, and the second information generation unit included in the information processing system may be included in the same apparatus, or may each be included in a different apparatus.

As described above, the information processing system may include a photographing unit such as a microscope or a camera, and the image data acquisition unit may thereby acquire image data, or image data acquired through photographing through use of a photographing apparatus outside the information processing system may be acquired through a network or the like by the image data acquisition unit. Further, the first information generation unit included in the information processing system may have a function of analyzing the image data acquired by the image data acquisition unit to generate the first information, or a configuration in which the image data is transmitted to an image analysis apparatus outside the information processing system and the image data acquisition unit included in the information processing system acquires the first information generated based on the analysis performed by the image analysis apparatus may be employed. Further, the display unit may be included in the information processing apparatus as described above, may be included in an apparatus different from the information processing apparatus included in the information processing system, or may be included in the information processing system as a stand-alone display apparatus.

### <Information Processing Method>

The present disclosure provides an information processing method including the steps of: acquiring image data including a colony of pluripotent stem cells; acquiring first information, which is information regarding a predetermined morphology of the colony of pluripotent stem cells, and has been generated by analyzing the acquired image data (based on analysis of the image data); and generating second information, which is information regarding chromosomal abnormalities in the pluripotent stem cells, based on the first information.

The present disclosure further provides a non-transitory storage medium storing a program that causes the above-mentioned information processing method.

### <Cell Manufacturing Method>

The present disclosure provides a cell manufacturing method including the steps of: acquiring image data including a colony of pluripotent stem cells; acquiring first information, which is information regarding a predetermined morphology of the colony, based on analysis of the acquired image data; generating second information, which is information regarding chromosomal abnormalities in the pluripotent stem cells, based on the acquired first information; and classifying the pluripotent stem cells being cultured based on the acquired second information.

The step of classifying the cells refers to, for example, performing classification into chromosomally abnormal cells, which are cells having abnormalities in chromosomes, and normal cells based on the second information, which is the information regarding the chromosomal abnormalities in the pluripotent stem cells.

Having abnormalities in chromosomes includes, as described later, excess, deletion, and modification of chromosomes, and having an abnormality in chromosomes refers to having at least one identified kind of chromosomal abnormality classified as any one of excess, deletion, or modification of chromosomes. Herein, having abnormalities in chromosomes may be referred to as being abnormal, a cell having an abnormality in chromosomes may be referred to as an abnormal cell, and a colony substantially formed of cells having abnormalities in chromosomes or a colony including cells having abnormalities in chromosomes may be referred to as an abnormal colony. Further, being normal does not always mean that no chromosomal abnormality is included, and refers to not having at least one identified kind of chromosomal abnormality or having no chromosomal abnormality detected. A cell not having at least one identified kind of chromosomal abnormality or having no chromosomal abnormality detected is referred to as a normal cell. Further, a colony that is substantially formed of normal cells and no abnormal cells is referred to as a normal colony.

The cell manufacturing method may further include a culturing step of selecting and culturing only a colony formed of normal cells based on classification results obtained in the step of classifying the cells. When the image data including a colony is acquired, a photographing position of the image is stored in the storage unit, and when the information regarding the chromosomal abnormalities is acquired, an analyzed position of the colony is associated therewith, thereby being able to grasp a positional relationship of the normal colony with respect to the image and to classify the colony.

In the culturing step, a commercially available apparatus such as a cell colony picking apparatus may be used to pick a normal colony and culture cells thereof.

For example, in a case of manufacturing cells through use of the above-mentioned information processing apparatus, it is also possible to manufacture cells by further connecting the information processing apparatus to a colony picking unit provided with a robot arm, a pump pipette, a moving belt, an incubator, and the like.

The present disclosure further provides a cell manufacturing device and or a cell manufacturing system including a first information acquisition unit configured to acquire, based on analysis of an image data including a colony of pluripotent stem cells, first information that is information regarding a predetermined morphology of the colony of pluripotent stem cells; a second information generation unit configured to generate, based on the first information, second information that is information regarding chromosomal abnormalities in the pluripotent stem cells, and classifying unit configured to classify pluripotent stem cells being cultured which include the pluripotent stem cells based on the second information.

### EMBODIMENT

One embodiment of the disclosure of the present specification is described below. In this embodiment, the following method is described: the information processing apparatus and the information processing method according to the present disclosure are used to acquire image data including a colony of pluripotent stem cells, acquire first information, which is information regarding a predetermined morphology of the colony, based on analysis of the acquired image data including the colony of pluripotent stem cells, and generate second information, which is information regarding chromosomal abnormalities in the pluripotent stem cells, based on the first information.

In general, normal iPS cells have characteristics of forming relatively rounded colonies, having a high nuclear-to-cytoplasmic ratio of each cell, and having the presence of a nucleus spreading throughout each entire cell. It is also known that the colonies are in a process of being formed while spreading for up to four days after seeding, and edges of the colonies are observed to be spiky and pointed. Meanwhile, in the colonies of iPS cells, irregularity in colony contour, cellular hypertrophy and heterogeneity within the colony, and the like are referred to as morphological abnormalities of the colony. It is known that morphological abnormalities in the colonies of iPS cells are associated with a decrease in undifferentiated markers, an increase in differentiation markers, karyotype abnormalities, or the like. In addition, in pluripotent stem cells, cells having chromosomal abnormalities exhibit changes in expression levels of E-cadherin involved in intercellular adhesion, and hence changes occur in the morphology of a colony formed of differentiated or cancerous cells. From such a background, the chromosomal abnormalities in pluripotent stem cells may affect the morphology of the colony. In this embodiment, a method of determining presence or absence of chromosomal abnormalities by focusing on such morphological characteristics of the colony of pluripotent stem cells is described with reference to FIG. 1.

(Step 1: Acquiring First Information, which is Information Regarding Predetermined Morphology of Colony of Pluripotent Stem Cells, and has been Generated by Analyzing Image Data)

As illustrated in FIG. 1, the flow using the information processing apparatus according to this embodiment includes Step 1 and Step 2.

Step 1 is a step of acquiring the first information, which is the information regarding the predetermined morphology of the colony of pluripotent stem cells, and has been generated by analyzing the image data. The information processing method according to this embodiment may include, as pre-steps of Step 1, a step of acquiring, by the image data acquisition unit, the image data including the colony of pluripotent stem cells and a step (first information generation step) of analyzing the acquired image data including the colony of pluripotent stem cells so as to generate the first information. The “analysis” as used herein is assumed to include analysis using a trained model as well. Thus, in Step 1, a trained model trained so as to output the first information, which is the information regarding the predetermined morphology of the colony, may be used to input thereto the image data including the colony of pluripotent stem cells and acquire the output first information, which is the information regarding the predetermined morphology of the colony of pluripotent stem cells. More specific examples of the trained model in this case include a regression model formed of a neural network based on, for example, a convolutional neural network (CNN). Examples of a method of training the trained model include a method involving constructing in advance training data in which the image data including the colony of pluripotent stem cells is associated with a scalar value or the like of a specific piece of first information corresponding to the image data as a label, and then performing machine learning through use of the training data, to thereby generate a trained model.

In the image including the colony of pluripotent stem cells, from which the first information is to be acquired in this step, the pluripotent stem cells are preferred to include iPS cells or ES cells and be cells that form a colony. Further, the colony refers to a cell aggregate in which a plurality of cells is gathered. In general, the colony refers to a cell population that has grown from a single cell, but sometimes cells other than the cell population that has grown from a single cell are mixed therein. The colony is preferred to have been cultured for 5 days to 8 days, have an outer circumferential length of 1,000 μm or more, or have a major axis length of 200 μm or more.

### (First Information)

The first information is the information regarding the predetermined morphology of the colony of pluripotent stem cells, which has been generated by analyzing the image data including the colony of pluripotent stem cells. The information regarding the predetermined morphology of the colony of pluripotent stem cells includes any information regarding the morphology of the colony of pluripotent stem cells, but can preferably be information regarding a morphology of an outer peripheral portion of the colony of pluripotent stem cells. The information regarding the morphology of the outer peripheral portion of the colony of pluripotent stem cells can include at least one of information regarding spikes present in the outer peripheral portion of the colony of pluripotent stem cells or information regarding a cell present in the outer peripheral portion of the colony of pluripotent stem cells.

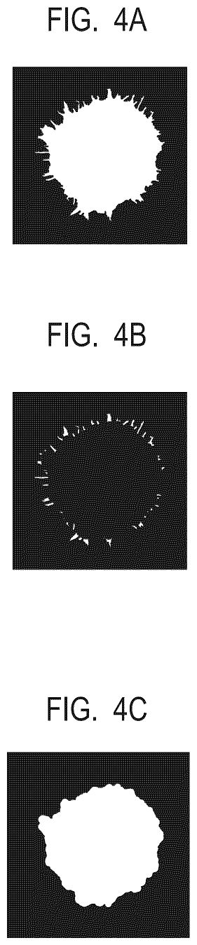

Further, the information regarding the spikes present in the outer peripheral portion of the colony of pluripotent stem cells can be information (for example, the number of spikes present in the outer peripheral portion of the colony of pluripotent stem cells) regarding the number of spikes present in the outer peripheral portion of the colony of pluripotent stem cells. The information regarding the number of spikes can also be analyzed through use of general image analysis software. The spike means a pointed shape or a sharp protrusion. FIG. 4A and FIG. 4B show an example of spike shapes. FIG. 4A shows a mask image of a colony before analysis, and FIG. 4B shows an example of a result of extracting spikes present in the outer peripheral portion of the colony through use of the image analysis software. For example, the image of the colony of pluripotent stem cells acquired by the image data acquisition unit is masked to create such a mask image as exemplified in FIG. 4A, and then the image analysis software is used to perform a top-hat operation on the mask image, to thereby be able to acquire such a spike image as exemplified in FIG. 4B. Counting the number of spikes extracted in this manner enables generation of information regarding the spikes present in the outer peripheral portion of the colony.

In order to distinguish spikes from minute protrusions or gentle convexities, the following method can be used. For example, the spikes may be extracted by binarizing the image including the colony of pluripotent stem cells and then removing a circular portion (particle portion) bounded at bases of the spikes.

The circular portion (particle portion) refers to a portion excluding the outer peripheral portion of the colony, and corresponds to, for example, a portion obtained by applying Opening processing on the mask image of the colony.

An example of the circular portion (particle portion) is shown in FIG. 4C. For example, a particle image shown in FIG. 4C can be created from the mask image of the colony before analysis shown in FIG. 4A through use of general image analysis software. After that, the particle image shown in FIG. 4C is subtracted from the mask image of the colony of pluripotent stem cells before analysis shown in FIG. 4A, to thereby be able to extract spikes as shown in FIG. 4B. All extracted images may be defined as spikes, or a threshold value may be determined by size to define spikes. For example, spikes may be defined as those having an area of 7.5 μm² or more, or may be defined as those having an area of 75 μm² or more. It is more preferred to define spikes as those having an area of 37 μm² or more. Further, the major axis length may be used as the threshold value in place of the area. Further, the threshold value may be set by circularity of the spike to remove rounded ones and consider the remaining ones as spikes.

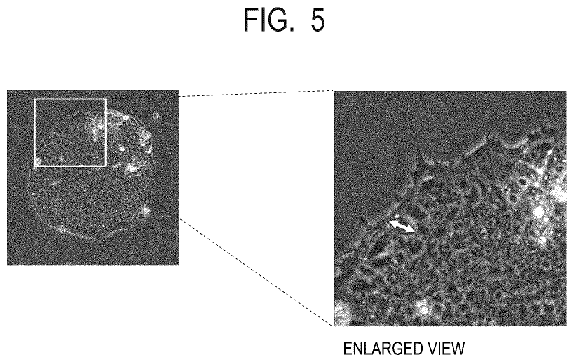

Further, the information regarding the cell present in the outer peripheral portion of the colony of pluripotent stem cells may be information regarding a cell present within a predetermined range from an outer periphery of the colony. Further, the information regarding the cell presents within the predetermined range from the outer periphery may be information regarding the nucleus of the cell present within the predetermined range from the outer periphery of the colony. The information regarding the nucleus of the cell can be information such as the size, area, circumferential length, major axis length, and minor axis length of the nucleus. Further, the predetermined range from the outer periphery may be set to, for example, a range within 50 μm from the outer periphery. The information regarding the nucleus of the cell may be obtained through use of existing image analysis software. FIG. 5 shows an image including a colony of pluripotent stem cells and an enlarged image of a part thereof, and a major-axis-direction length of a cell present near the outer periphery of the colony is indicated by the arrow. As in this example, the major-axis-direction length of the cell present near the outer periphery may be measured to obtain the information regarding the nucleus of the cell. The number of cells to be measured may be one, or a plurality of cells may be measured and an average value thereof may be calculated.

(Step 2: Generating Second Information, which is Information Regarding Chromosomal Abnormalities in Pluripotent Stem Cells, Based on First Information)

Step 2 is a step of generating the second information, which is the information regarding the chromosomal abnormalities in the pluripotent stem cells, based on the first information, which is the information regarding the predetermined morphology of the colony, and has been generated by analyzing the image data including the colony and acquired in Step 1.

(Second Information, which is Information Regarding Chromosomal Abnormalities in Pluripotent Stem Cells)

The information regarding the chromosomal abnormalities in the pluripotent stem cells may include at least one of information regarding a determination result of whether or not the pluripotent stem cells include chromosomally abnormal cells, information regarding a proportion of the chromosomally abnormal cells included in the pluripotent stem cells, or information indicating a type of a chromosomal abnormality included in the pluripotent stem cells. For example, determination may be performed as to types of chromosomal abnormalities such as “deletion,” “duplication,” “inversion,” “translocation” or “normal.” In particular, it is known that abnormalities in chromosomes 1, 12, 17, and 20 frequently occur in human pluripotent stem cells such as iPS cells or ES cells, and those abnormalities are also considered to relate to cancerization and cancer progression, which has become a problem. Thus, the information regarding the chromosomal abnormalities may be information regarding chromosomes 1, 12, 17, and 20. The information regarding the chromosomal abnormalities are more preferred to be information regarding a chromosome 12 abnormality, and specifically, information regarding an excessive copy number of chromosome 12, that is, trisomy 12. The information regarding the determination result of whether or not the pluripotent stem cells include chromosomally abnormal cells may be the determination result itself, or may be an image showing a portion including chromosomally abnormal cells in the image including the colony. The image showing the portion including chromosomally abnormal cells in the image including the colony may be, for example, an image showing a region determined to include chromosomally abnormal cells in the image including the colony, such as an image obtained by superimposing the image including the colony and an image showing a region determined to include chromosomally abnormal cells. Further, positions of the abnormal cells may be calculated through use of an analyzed coordinate position of the colony recorded in the storage unit, and used as the information regarding the determination result. Further, when abnormal cells are detected in a specific well in a culture vessel, a well in which abnormal cells have occurred and a normal well may be determined on a per-well basis, and used as the information regarding the determination result.

An apparatus different from the information processing apparatus according to this embodiment may generate the first information by performing a step of acquiring image data or a step of analyzing the image data, and the generated first information may be acquired by the information processing apparatus according to this embodiment, thereby forming Step 1. Alternatively, the information processing apparatus according to this embodiment may perform the step of acquiring image data or the step of analyzing the image data to generate and acquire the first information. In the former case, for example, the first information may be input to the information processing apparatus according to this embodiment through an input unit to acquire the first information, and the second information generation unit may generate the second information through use of the acquired information.

When the second information generation unit generates the second information, a threshold value regarding the first information may be set, and the second information may be generated based on the threshold value. The threshold value may be obtained by an arithmetic operation unit based on a trained model, or information regarding a correspondence relationship between the first information and the second information, which is described later. Alternatively, as the threshold value, a value determined in advance may be input by an operator through the input unit. Examples of the threshold value may include a value relating to the number of spikes of the colony and the size, area, circumferential length, major axis length, and minor axis length of the nucleus of the cell present within the predetermined range from the outer periphery of the colony. For example, according to the following Example, as a preferred example of the threshold value, the threshold value of the number of spikes can be set to 25 so that a colony having 25 or more spikes is determined to be normal and a colony having fewer than 25 spikes is determined to have chromosomal abnormalities.

Further, in addition to the first information, third information, which is information of a different type from that of the first information, may be used, and threshold values regarding the first information and the third information may be provided to generate the second information. A specific example of the third information is described later.

As described above, the second information may be generated by inputting the first information to a trained model. The trained model can be constructed as follows. Examples of a model that generates the second information from the first information include a determination model formed of a neural network based on, for example, a convolutional neural network (CNN). Examples of the method of training a trained model in this case include a method involving constructing in advance training data in which the scalar value of the first information extracted from the image data on the colony of pluripotent stem cells by the above-mentioned method is used as input to be associated with types of chromosomal abnormalities of the corresponding colony of pluripotent stem cells as a label, and then performing machine learning through use of the training data, to thereby generate a trained model. Further, when the threshold value regarding the first information is acquired by the trained model, examples of the model include a decision tree and a support vector machine (SVM). Those models are machine learning algorithms that estimate a threshold value for classifying an estimated class for an input scalar value. As with the above-mentioned neural network learning, it is possible to construct a trained model that estimates the threshold value regarding the first information for acquiring the second information by performing pre-training through use of the training data in which the scalar value of the first information extracted from the image data on the colony of pluripotent stem cells is associated with types of chromosomal abnormalities or the like of the corresponding colony of pluripotent stem cells as a label. Pieces of data including images including colonies of pluripotent stem cells having chromosomal abnormalities and images including colonies of pluripotent stem cells having no chromosomal abnormality together with the information regarding the chromosomal abnormalities are prepared, and after acquisition of the first information, those pieces of training data are used to obtain a trained model that uses the first information as input and generates information regarding the chromosomal abnormalities which is the second information.

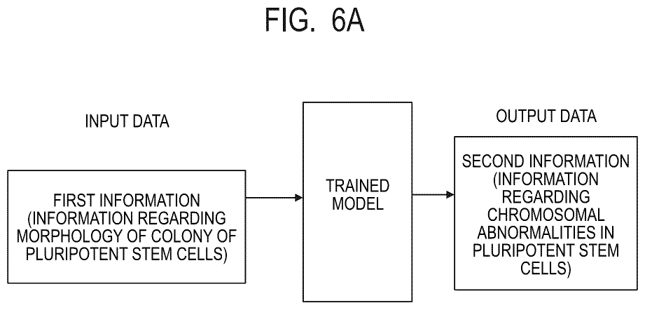

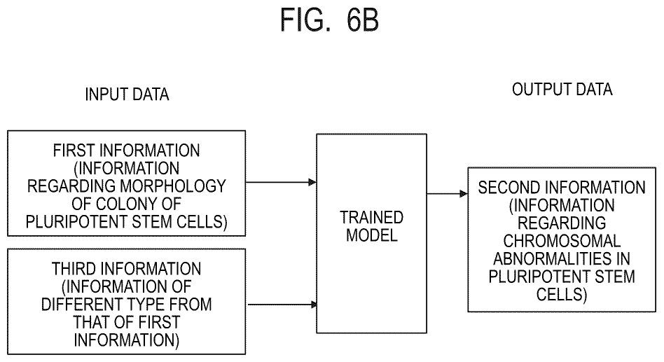

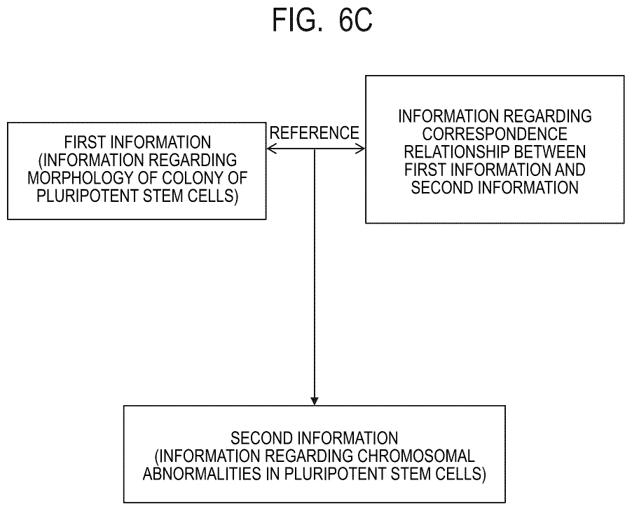

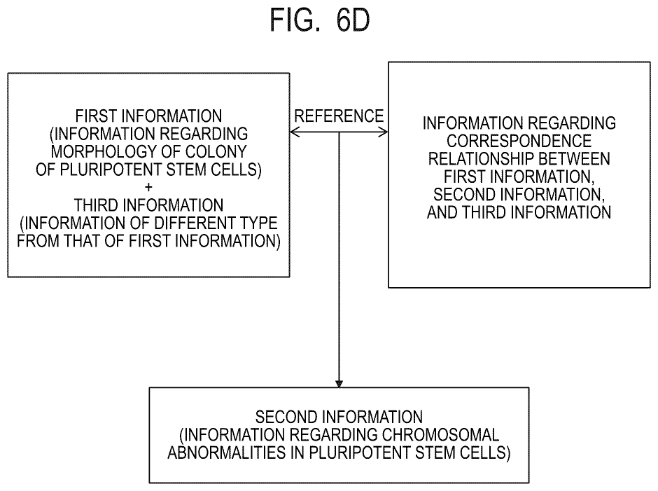

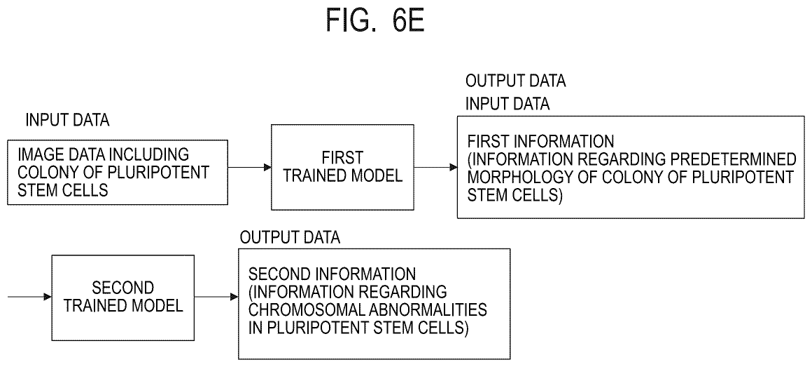

As illustrated in FIG. 6A, the first information can be input to such a trained model to generate the second information. Further, as illustrated in FIG. 6E, the image data including the colony of pluripotent stem cells can be input to a first trained model to generate the first information, and the generated first information can be input to a second trained model to generate the second information, which is the information regarding the chromosomal abnormalities in the pluripotent stem cells. In such a case, the first trained model and the second trained model are different models.

The trained model may be obtained through training by inputting not only the first information but also the third information, which is the information of a different type from that of the first information. A flow in a case of using the trained model obtained in this manner is illustrated in FIG. 6B. As illustrated in FIG. 6B, the first information, which is the information regarding the predetermined morphology of the colony in colony image data, and has been acquired by the first information acquisition unit, and the third information, which is the information of a different type from that of the first information, and has been acquired in advance, can be input to the trained model to generate the second information, which is the information regarding the chromosomal abnormalities in the pluripotent stem cells.

The trained model is a model obtained by training in advance a machine learning model in accordance with a freely-selected machine learning algorithm such as deep learning. The trained model has been trained in advance, which does not mean that no further training is to be performed, and additional training may be performed. Additional learning may be performed at any time based on the data obtained when the information processing method according to this embodiment is executed.

The trained model may be a regression equation obtained by regression analysis. Further, the trained model may be formed of a combination of a plurality of multiple regression equations, or may be formed of a single multiple regression equation.

In another case, as illustrated in FIG. 6C, information regarding the correspondence relationship between the first information and the second information acquired in advance may be stored in the storage unit or the like, and the second information may be generated based on the input first information by referring to the information regarding the correspondence relationship. Further, as illustrated in FIG. 6D, the third information, which is the information of a different type from that of the first information, may be used to store information regarding a correspondence relationship between the first information, the second information, and the third information in the storage unit or the like, and the second information may be generated based on the input first information and third information by referring to the information regarding the correspondence relationship.

### (Third Information)

The third information is the information of a different type from that of the first information, and is preferred to include at least one of the following: culture conditions for the pluripotent stem cells; a type of a line of the pluripotent stem cells; or information regarding a predetermined morphology of a colony serving as a comparison target which has been acquired from an image including the colony serving as the comparison target. For example, the culture conditions can include a condition regarding at least any one of pieces of information on a cell seeding density, a culture vessel, a type and concentration of a coating agent, the number of culture days, or the number of passages. As the comparison target, it is possible to use standard cells that have been determined to be normal (determined to have no chromosomal abnormality detected) or abnormal (determined to include chromosomal abnormalities) in advance by another test. For example, a colony of cells known to be normal in advance can be used as the comparison target, and when the information regarding the predetermined morphology of the colony in the colony of normal cells is input, comparison with that value enables acquisition of the second information. Meanwhile, the comparison target may be a colony that has been determined to be abnormal in place of the colony of normal cells. In general, the morphology of the colony is affected by several factors such as the number of culture days since the last passage, a used cell line, a passage method, and a used matrix, and thus, it is preferred to culture cells to be evaluated and comparison cells (standard cells) under the same conditions and acquire images of the colonies under the same conditions.

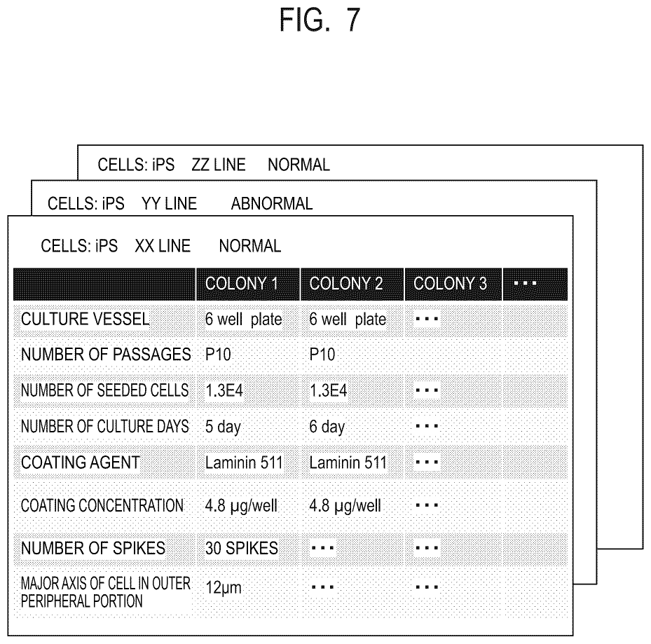

In FIG. 7, an example of the first information and the third information acquired in advance is illustrated. In this example, the number of spikes and the major axis of the cell in the outer peripheral portion for each colony of pluripotent stem cells are used as the first information, and the culture vessel, the number of passages, the number of seeded cells, the number of culture days, the coating agent, and a coating concentration are used as the third information. Those are listed together with the type and name of cells and information (second information) on whether or not the cells are normal. For example, such data as illustrated in FIG. 7 is stored in advance in the storage unit, and the third information input by the operator and the data stored in the storage unit are compared, to thereby enable a threshold value for generating the second information to be determined by referring to the first information of the colony of pluripotent stem cells in a case of being cultured under the same conditions or similar conditions. The threshold value may be calculated and determined through use of the information processing apparatus, or may be determined by being input by the operator with reference to the first information.

The data illustrated in FIG. 7 is merely an example indicating sheets for summarizing data by type of cells, but, for example, sheets for summarizing the first information by number of culture days or sheets for summarizing the first information by change in number of passages may be used.

The second information is displayed on the display unit under the control of a display control unit included in the information processing apparatus. This may be performed, at a timing set in advance or when an instruction reception unit included in the information processing apparatus receives an operation from the operator, by the information processing apparatus automatically controlling a flow in which the image data acquisition unit acquires the image data including the colony, the first information is acquired by analyzing the acquired image data, and the display control unit displays the second information generated based on the first information on the display unit. The information to be output may be the second information or may be information generated from the second information. Those may be collectively referred to as information based on the second information.

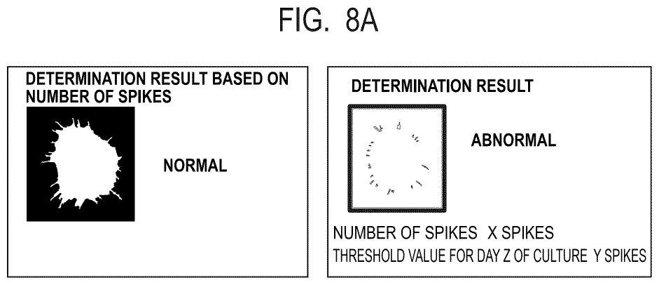

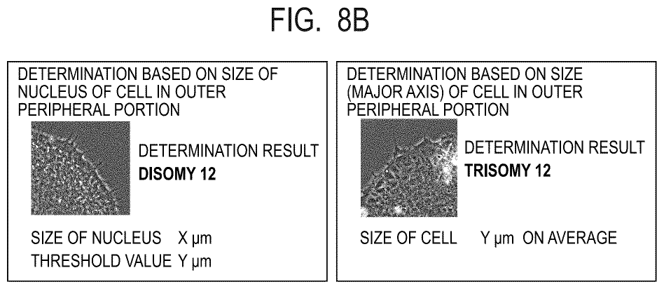

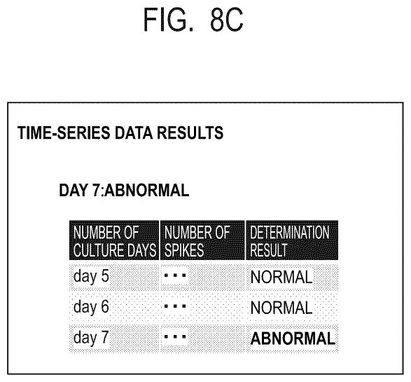

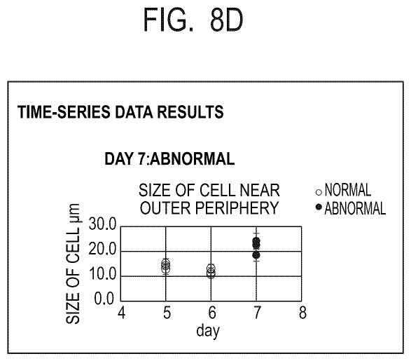

Examples of display in a case of outputting the second information are illustrated in FIG. 8A to FIG. 8D.

FIG. 8A is an example of display screens in a case of using the number of spikes as the first information (information regarding the predetermined morphology of the colony of pluripotent stem cells). An example of a normal case is indicated on the left, and an example of an abnormal case is indicated on the right.

FIG. 8B is an example of display screens in a case of using a size of a cell present in the outer peripheral portion as the first information. An example in which the determination result is disomy 12 is illustrated on the left, and an example in which the determination result is trisomy 12 is illustrated on the right. In generating the second information, the determination result may be displayed so as to indicate, among pieces of information regarding the morphology of the colony of pluripotent stem cells, which type of morphology information has been used to acquire the first information, or only the determination result of “abnormal” or “normal” may be displayed. Further, when the determination is difficult, an inconclusive determination (may be called as “a gray determination”, or “an intermediate determination”) may be made. Further, whether or not chromosomal abnormalities are present in the input image may be output. Further, a representative image indicating predetermined morphological characteristics of the colony of pluripotent stem cells used for the determination may be displayed, or the number of the morphological characteristics or the threshold value used for the determination may be displayed.

Further, in a case of acquiring time-series data, a table including the number of culture days and the first information may be displayed together with the second information as illustrated in FIG. 8C, or information regarding at least one of the number of culture days, the number of passages, or the size of the colony may be displayed together with the second information.

The time-series data may be displayed as a graph as in FIG. 8D. In this case, the change by number of days is indicated as a time series, but the number of passages or a change over time may be indicated.

Further, when an index such as a cursor is placed on partial data (such as a cell in a table or a plot in a graph) of the displayed time-series data, the image including the colony of pluripotent stem cells used in calculating the first information or the representative image indicating the characteristics of the first information may be displayed. The image itself including the colony of pluripotent stem cells may be displayed, or an enlarged image of the outer peripheral portion of the colony may be displayed. For example, the image of spikes of the colony of pluripotent stem cells may be displayed as in FIG. 4A or FIG. 4B, or the enlarged image of the outer peripheral portion may be displayed as in FIG. 5.

### Modification Example 1

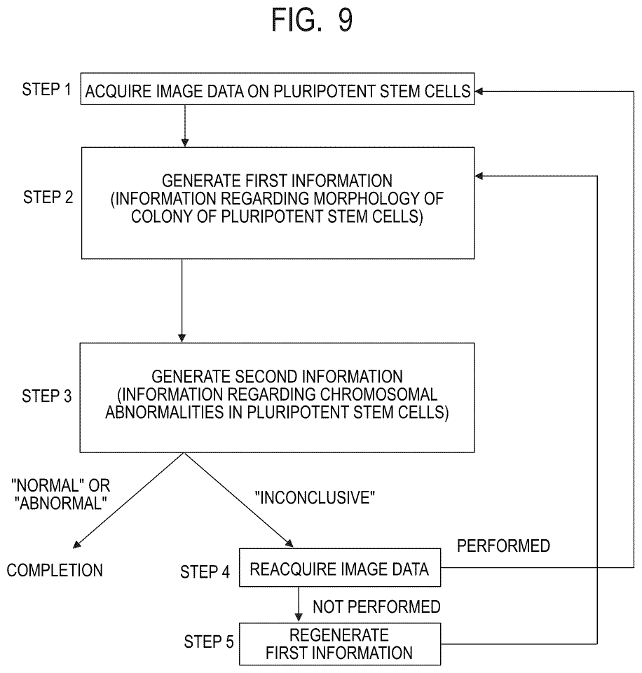

As Modification Example 1, a case in which the information processing apparatus and the information processing method according to the present disclosure are used to acquire the first information, which is the information regarding the predetermined morphology of the colony, from the image including the colony of pluripotent stem cells and generate second information, which is the information regarding the chromosomal abnormalities in the pluripotent stem cells, based on the acquired first information, resulting in an inconclusive determination is described. An example of a flow of performing the analysis again when the second information results in an inconclusive determination is illustrated in FIG. 9.

In this embodiment, the image data including the colony of pluripotent stem cells is acquired as Step 1, and as Step 2, the image data including the colony of pluripotent stem cells is analyzed to generate and thereby acquire the first information, which is the information regarding the predetermined morphology of the colony. After that, the second information, which is the information regarding the chromosomal abnormalities in the pluripotent stem cells is generated based on the first information as Step 3. Step 2 and Step 3 are separately described here, but can also be carried out as one step. The inconclusive determination refers to, for example, a case in which it is difficult to evaluate whether the result is “normal” or “abnormal” (for example, a case in which information based on the first information is within a predetermined range), and in that case, as indicated in Step 4, the operator may select whether or not to reacquire the image data.

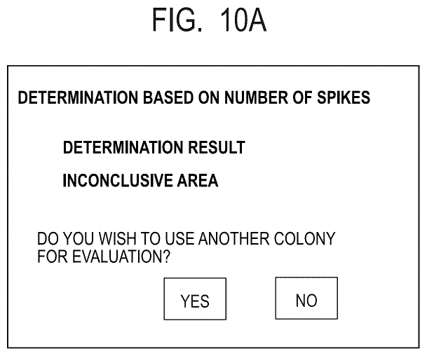

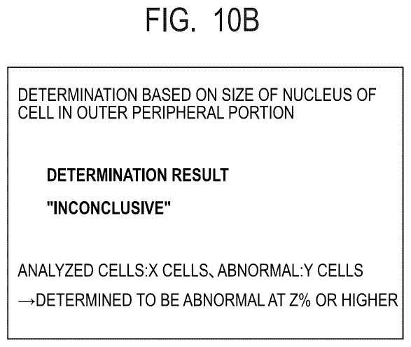

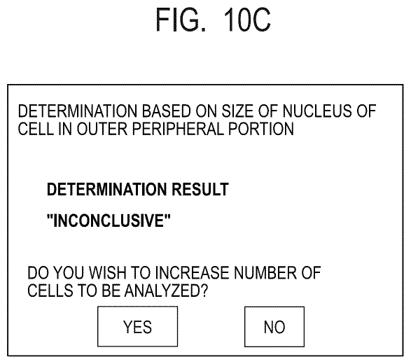

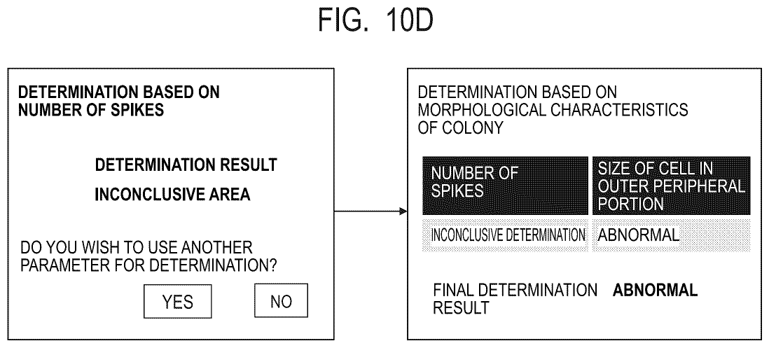

In FIG. 10A to FIG. 10D, examples of screens displayed on the display unit by the display control unit when the determination is difficult are illustrated. The fact that the determination is difficult may be displayed as, for example, “INCONCLUSIVE AREA” as illustrated in FIG. 10A and on the left of FIG. 10D, or “INCONCLUSIVE” as illustrated in FIG. 10B and FIG. 10C, or other representations may be used. The inconclusive determination may be displayed as “unable to determine.” In addition, as illustrated in FIG. 10A, in FIG. 10C, and on the left of FIG. 10D, for example, a message that prompts the operator to perform redetermination through use of a different parameter may be displayed.

In a case of reacquiring the image data including the colony, it is possible to return from Step 4 to Step 1 of FIG. 9 to reacquire image data of a different field of view, or to reacquire image data of the same field of view under different conditions. Meanwhile, in a case of not reacquiring the image data, it is also possible to regenerate the first information as indicated in Step 5. In this case, it is also possible to regenerate the first information through use of information regarding a morphology of a different type from that of the information regarding the morphology used when an inconclusive determination is made. For example, when the information on the size of a cell in the outer peripheral portion has been used as the first information in the case of an inconclusive determination, it is also possible to perform Step 2 again through use of the number of spikes as the first information. It is also possible to perform Step 2 again by, for example, taking measures such as increasing the number of cells to be counted without changing the first information.

In regard to the display of the second information, in the case of an inconclusive determination as illustrated in FIG. 10B, when the determination has been performed based on the size of the nucleus of the cell, the number of analyzed cells and the number and proportion of abnormal cells may be displayed. In another example, as illustrated in FIG. 10C, such display as to prompt the user to select whether or not to increase the number of cells to be analyzed may be performed. In addition, Step 2 may be performed again through use of a plurality of types of first information (a plurality of types of information regarding the morphology of the colony of pluripotent stem cells) such as the number of spikes and the size of a cell in the outer peripheral portion. As illustrated in the display on the left side of FIG. 10D, in the case of an inconclusive determination after generation of the second information through use of the number of spikes in the colony as the first information, information for receiving an instruction to determine whether or not to perform the determination again based on another parameter may be displayed. Such display as to prompt the user to select “YES” or “NO” may be performed, or, for example, the display may be performed so that which parameter is to be used to perform the determination again can be selected from a pull-down menu (that is, a GUI that allows selection of the type of first information to be used to generate or acquire the second information may be displayed). On the right of FIG. 10D, an example of display performed when the determination results in “abnormal” after selection of performing the determination again through use of the size of the cell present in the outer peripheral portion of the colony as the first information in response to the display on the left of FIG. 10D is illustrated. A first determination result and a second determination result may be displayed side by side, and then a final determination result may be displayed as well, or only the final determination result may be displayed.

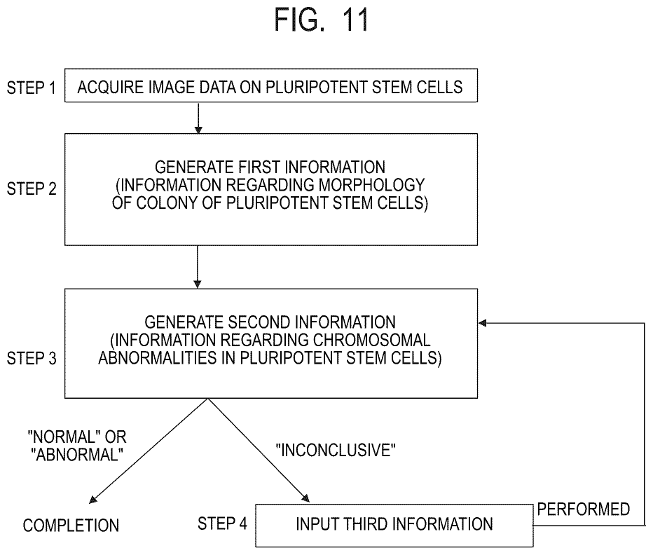

Such a flow as illustrated in FIG. 11 may be carried out. The processing steps of up to Step 3 are the same as those described above. In the flow illustrated in FIG. 11, after an inconclusive determination is made in Step 3, the third information is input as indicated in Step 4, to thereby perform Step 2 and Step 3 again, that is, regenerate the first information and the second information. The third information can be parameter information, for example, can be information on the number of culture days. In this manner, the third information is added to perform Step 3 again, to thereby be able to perform the determination by referring to data on cells that have been cultured for a similar number of days. Further, when information regarding the size of the colony of pluripotent stem cells to be used for the determination, such as the circumferential length or the major axis length, is input as the third information, data on the colony of pluripotent stem cells which has the same size can be referred to.

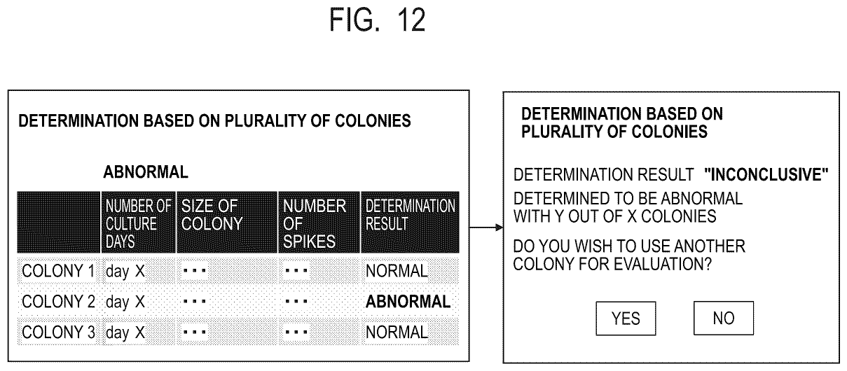

Further, to generate the second information, it is also possible to use the first information on a plurality of colonies of pluripotent stem cells. For example, it is also possible to display such a screen as illustrated on the left of FIG. 12 on the display unit, input information regarding morphologies of the plurality of colonies generated by analyzing the acquired image data (which may include a plurality of images) as the first information, and determine whether or not there is a chromosomal abnormality for each colony of pluripotent stem cells. In this case, the determination may be performed through use of the trained model and the information regarding the correspondence relationship by inputting not only the first information but also the third information such as the number of culture days of each colony and the size of the colony. When even one of the colonies is determined to be abnormal, the determination may result in “abnormal,” or when the proportion of colonies determined to be abnormal is small, such a screen as illustrated on the right of FIG. 12 may be displayed on the display unit so as to indicate that the determination results in “INCONCLUSIVE.” Further, when the proportion of colonies determined to be abnormal exceeds a certain threshold value (reference value), the determination result may be displayed as “abnormal.” Further, the number of analyzed colonies of pluripotent stem cells and the number and proportion of colonies determined to be abnormal may be displayed. Further, the display may be performed so that a display form such as a text color may differ between a case of the determination resulting in “normal” and a case of the determination resulting in “abnormal,” such as blue for “normal,” red for “abnormal,” and yellow for an inconclusive determination.

### Modification Example 2

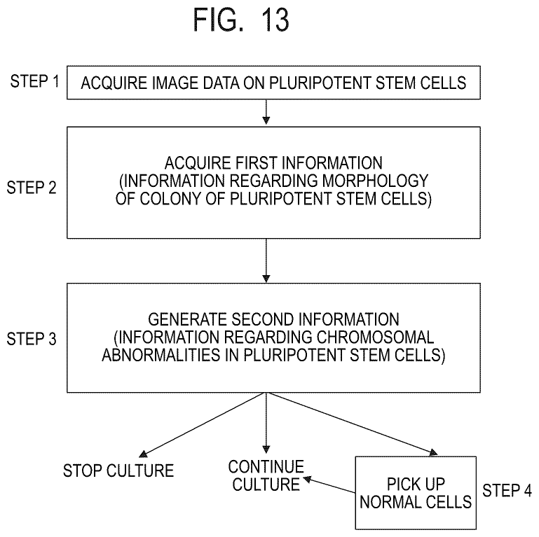

As Modification Example 2, a case in which a cell manufacturing method according to the present disclosure is used to culture pluripotent stem cells is described. An example of a flow therefor is illustrated in FIG. 13. The processing steps of up to Step 3 are the same as those described above. In the example of FIG. 13, when the determination results in “normal” in Step 3, the culture may be continued as is, or, as Step 4, that colony may be picked up and the culture may be continued. Meanwhile, when the determination results in “abnormal” in Step 3, the culture can also be stopped. Information for receiving an instruction to select whether or not to continue the culture or whether or not to pick up the colony may be displayed on the display unit together with the second information, and the operator may perform the selection.

Further, programming may be performed in advance so that the colony is picked up when the colony is determined to be normal. When it is desired to continue the culture even in the case of the determination resulting in “abnormal” or “gray,” the determination may be performed again as in Modification Example 1 to find a colony determined to be normal, and then the process may advance to Step 4 to pick up that colony.

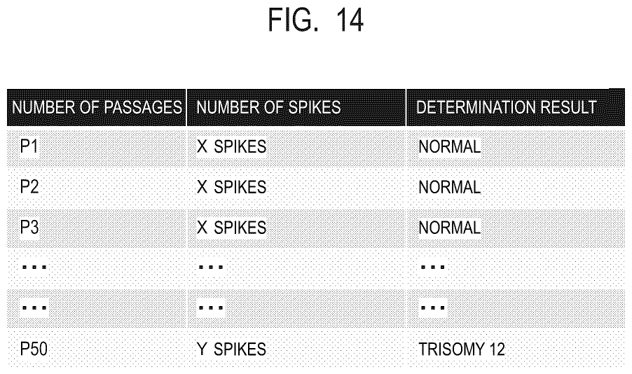

For example, a cell culture apparatus in this embodiment may be used to continuously examine whether or not chromosomal abnormalities have occurred in cells being cultured. It is also possible to acquire the image data including the colony of pluripotent stem cells at the same number of culture days set for each passage, and observe progress thereof. As illustrated in FIG. 14, the number of passages serving as the third information different from the information regarding the morphology of the colony, the number of spikes present in the outer peripheral portion of the colony serving as the first information, and the determination result of “normal” or “abnormal” serving as the second information may be stored in the storage unit, and the second information such as the determination result as to whether or not abnormalities have occurred may be generated from changes in the first information and the determination result through use of the trained model and the information regarding the correspondence relationship. It is also possible to store only the third information different from the information regarding the morphology of the colony and the second information. Further, data acquired in advance can be used to determine the threshold value for generating the second information and store the determined threshold value. The threshold value may be determined by carrying out a significance test between the abnormal and normal cases in the first information through use of the data acquired in advance. The determination may result in “abnormal” when an outlier occurs with respect to the first information, which has been generated by analyzing normal colonies and acquired in advance. The outlier can be determined by a method such as determination using a standard deviation, determination using a box-and-whisker plot, or cluster analysis.

For example, a criterion therefor may be whether or not a given value is more than three times a value of the standard deviation, and any value that is more than three times the value of the standard deviation may be considered to be abnormal. Alternatively, the box-and-whisker plot may be used, and any value that falls out of the range of whiskers may be considered to be abnormal.

Further, when the trained model is used to generate the second information, it is possible to detect that abnormalities have occurred in the cells being cultured. For example, the screen illustrated in FIG. 14 is an example in which the determination resulting in “abnormal” is displayed when the number of passages is 50. At this time, the operator can determine whether or not to continue the culture. To continue the culture even when the determination results in “abnormal,” Step 1 to Step 3 of the flow illustrated in FIG. 13 are performed again to find a colony formed of normal cells, and that colony is picked up in Step 4, to thereby be able to continue the culture of the cells.

### EXAMPLES

A more specific description is given below with reference to Examples. The embodiment of the present disclosure is not limited to the following Examples.

In this Example, an iPS cell line for research was used. A protocol for the culture followed the protocol “Human iPS cell culture under feeder-free conditions” published by CiRA-F.

In this Example, a 6-well plate pre-coated with Laminin 511-E8 diluted to 4.8 μg/well in accordance with the protocol was used as the culture vessel. In accordance with the protocol, iPS cells cultured for 7 days were detached and collected, and 13,000 living cells were seeded onto the pre-coated 6-well plate and cultured in an incubator at 37° C. with 5% CO₂ concentration. On the following day, the medium was replaced by a maintenance culture medium (StemFit AK03N) that did not contain Y-27632, and was thereafter replaced once every two days, and the culture was continued until the next passage.

### Example 1

In this Example, a case of determining in advance the threshold value for the first information to be used to generate the second information is described. In order to determine the threshold value, cells determined in advance to have trisomy 12 or disomy 12 were cultured to acquire data. In Step 1, image data including the colony was acquired from day 5 to day 9 of culture through use of a BZ-X810 manufactured by Keyence Corporation.

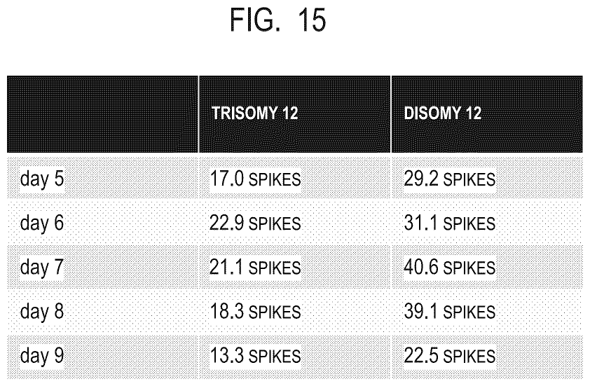

Subsequently, the number of spikes of the colony in the image data including the colony obtained in Step 1 was analyzed through use of ImageJ, to thereby generate and acquire the data illustrated in FIG. 15. The values indicated in FIG. 15 are average numbers of spikes obtained when 5 or more colonies were analyzed. The number of spikes obtained in this manner, the number of culture days, the number of passages and type of cells, and the like are stored in advance in the apparatus, and can thus be used to generate the second information. In this Example, the numbers of spikes were counted and results thereof were stored, but image data may be stored and used in place of numerical information.

In this Example, cells to be determined were cultured, and on day 5, the image data including the colony was acquired as Step 1, and was analyzed to find that the average number of spikes was 31. In this case, in Step 2, the apparatus can display the numbers of spikes of trisomy 12 and disomy 12 on day 5 from among the values indicated in FIG. 15, and the number of spikes being 31 is determined to indicate disomy 12.

### Example 2

In this Example, a case of simultaneously culturing evaluation cells and standard cells is described. The standard cells as used herein refers to cells that have been determined to be normal or abnormal in advance by the G-band analysis or the like. In this Example, cells that have been determined to be normal by the G-band analysis are used as the standard cells. The evaluation cells in this Example refers to iPS cells being cultured, which are target cells to be examined as to whether or not trisomy 12 has occurred.

The standard cells and the evaluation cells were cultured under the same conditions, and images including the colonies were acquired in Step 1.

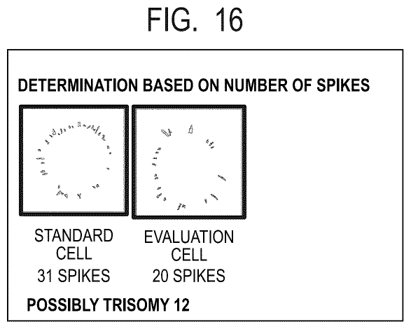

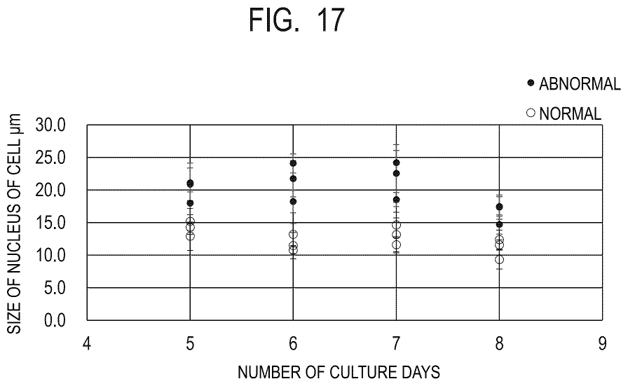

In this Example, the images were acquired on day 6 of culture, and the second information was acquired from the number of spikes in Step 2. An example of display of a result thereof is illustrated in FIG. 16. In this Example, the number of spikes was 31 for the standard cells, and was 20 for the evaluation cells. From the data acquired in advance and illustrated in FIG. 15, the threshold value for “normal” or “abnormal” of the colony cultured under the culture conditions in this Example was set to 25, and hence the possibility of trisomy 12 was indicated in this Example.

### Example 3

In this Example, a case in which the size of the cell present in the outer peripheral portion of the colony was used as the first information for generating the second information is described. In the same manner as in Example 1, in order to determine the threshold value in advance, cells that have been determined in advance to have trisomy 12 or disomy 12 were cultured to acquire data.

First, in Step 1, image data including the colony was acquired from day 5 to day 9 of culture through use of the BZ-X810 manufactured by Keyence Corporation. Subsequently, the size of the nucleus of the cell presents in the outer peripheral portion of the image of the colony acquired in Step 1 was measured through use of the ImageJ. Results thereof are shown in FIG. 17. The size of the nucleus obtained in this manner, the number of culture days, the number of passages and type of cells, and the like are stored in advance in the apparatus, to thereby be able to be used to generate the second information. Further, in this Example, the major axis length of each nucleus was measured and results thereof were stored, but images may be stored and used in place of numerical information. In this Example, the threshold value for “normal” or “abnormal” was set to 15 μm with reference to FIG. 17 to be used for the determination.

In this Example, the cells to be determined were cultured, and on day 7, the image including the colony was acquired as Step 1, and was analyzed to find that the size of the nucleus was 20 μm. In this case, in Step 2, the cells were determined to be abnormal.

According to the present disclosure, it is possible to perform a test regarding the chromosomal abnormalities in pluripotent stem cells even on cells that require non-invasive testing, such as cells being cultured.

The disclosure of this embodiment has the following configurations and methods.

### (Configuration 1)

An information processing apparatus including:
a first information acquisition unit configured to acquire, based on analysis of image data including a colony of pluripotent stem cells, first information that is information regarding a predetermined morphology of the colony of pluripotent stem cells; and
a second information generation unit configured to generate, based on the first information, second information that is information regarding chromosomal abnormalities in the pluripotent stem cells.

### (Configuration 2)

The information processing apparatus according to Configuration 1, wherein the first information is information regarding a morphology of an outer peripheral portion of the colony of pluripotent stem cells.

### (Configuration 3)

The information processing apparatus according to Configuration 2, wherein the information regarding the morphology of the outer peripheral portion of the colony of pluripotent stem cells includes at least one of information regarding spikes present in the outer peripheral portion of the colony of pluripotent stem cells or information regarding a cell present in the outer peripheral portion of the colony of pluripotent stem cells.

### (Configuration 4)

The information processing apparatus according to Configuration 3, wherein the information regarding the spikes present in the outer peripheral portion of the colony of pluripotent stem cells is information regarding the number of spikes present in the outer peripheral portion of the colony of pluripotent stem cells.

### (Configuration 5)

The information processing apparatus according to Configuration 3 or 4, wherein the information regarding the cell present in the outer peripheral portion of the colony of pluripotent stem cells is information regarding a cell present within a predetermined range from an outer periphery of the colony of pluripotent stem cells.

### (Configuration 6)

The information processing apparatus according to Configuration 5, wherein the information regarding the cell present within the predetermined range from the outer periphery of the colony of pluripotent stem cells is information regarding a nucleus of the cell present within the predetermined range from the outer periphery of the colony of pluripotent stem cells.

### (Configuration 7)

The information processing apparatus according to Configuration 6, wherein the information regarding the nucleus of the cell present within the predetermined range from the outer periphery of the colony of pluripotent stem cells includes at least one of information regarding a size, information regarding an area, information regarding a circumferential length, information regarding a major axis length, or information regarding a minor axis length, of the nucleus of the cell present within the predetermined range from the outer periphery of the colony of pluripotent stem cells.

### (Configuration 8)

The information processing apparatus according to any one of Configurations 1 to 7, wherein the second information includes at least one of information regarding a determination result of whether the pluripotent stem cells include chromosomally abnormal cells, information regarding a proportion of the chromosomally abnormal cells included in the pluripotent stem cells, or information indicating a type of a chromosomal abnormality included in the pluripotent stem cells.

### (Configuration 9)

The information processing apparatus according to any one of Configurations 1 to 8, further including:
an image data acquisition unit configured to acquire the image data; and
a first information generation unit configured to generate the first information by analyzing the image data acquired by the image data acquisition unit.

### (Configuration 10)

The information processing apparatus according to any one of Configurations 1 to 9, wherein the second information generation unit is configured to generate the second information based on the first information and third information which is different in type from the first information acquired in advance.

### (Configuration 11)

The information processing apparatus according to Configuration 10, wherein the third information includes at least one of information regarding culture conditions for the pluripotent stem cells, information regarding a type of a line of the pluripotent stem cells, or information regarding a predetermined morphology of a colony of pluripotent stem cells serving as a comparison target which has been acquired from the image data including the colony of pluripotent stem cells serving as the comparison target

### (Configuration 12)

The information processing apparatus according to any one of Configurations 1 to 11, wherein the second information is information regarding a chromosome 12 abnormality in the pluripotent stem cells.

### (Configuration 13)

The information processing apparatus according to any one of Configurations 1 to 9, further including a photographing unit configured to pick up an image including the colony of pluripotent stem cells.

### (Configuration 14)

The information processing apparatus according to any one of Configurations 1 to 13, wherein the first information acquisition unit is configured to acquire the first information generated by inputting the image data including the colony of pluripotent stem cells to a first trained model obtained through training based on training data including the image data including the colony of pluripotent stem cells and a scalar value of the first information serving as a label corresponding to the image data.

### (Configuration 15)

The information processing apparatus according to any one of Configurations 1 to 14, wherein the second information generation unit is configured to generate the second information by inputting the first information to a second trained model obtained through training based on training data including: a scalar value of the first information that is the information regarding the predetermined morphology of the colony of pluripotent stem cells; and types of chromosomal abnormalities of the colony of pluripotent stem cells serving as a label corresponding to the scalar value.

### (Configuration 16)

The information processing apparatus according to any one of Configurations 1 to 14, wherein the second information generation unit is configured to refer to information regarding a correspondence relationship between the first information and the second information to generate the second information from the first information.

### (Configuration 17)

The information processing apparatus according to any one of Configurations 1 to 16, wherein the second information generation unit is configured to generate the second information based on a predetermined threshold value that is based on the information regarding the predetermined morphology of the colony of pluripotent stem cells.

### (Configuration 18)

The information processing apparatus according to any one of Configurations 1 to 17, further including a display control unit configured to perform control to display the second information on a display unit.

### (Configuration 19)

The information processing apparatus according to Configuration 18, wherein the display control unit is configured to perform control to display the first information together with the second information on the display unit.

### (Configuration 20)

The information processing apparatus according to Configuration 18 or 19, wherein the display control unit is configured to perform control to display time-series data of the second information on the display unit.

### (Configuration 21)

The information processing apparatus according to Configuration 20, wherein the display control unit is configured to perform control to display, on the display unit, information regarding at least one of the numbers of culture days, the number of passages, or a size of the colony, of the pluripotent stem cells together with the time-series data of the second information.

### (Configuration 22)

The information processing apparatus according to any one of Configurations 18 to 21, further including the display unit.

### (Configuration 23)

An information processing system including:
an image data acquisition unit configured to acquire image data including a colony of pluripotent stem cells;
a first information acquisition unit configured to acquire, based on analysis of the image data, first information that is information regarding a predetermined morphology of the colony of pluripotent stem cells; and
a second information generation unit configured to generate, based on the first information, second information that is information regarding chromosomal abnormalities in the pluripotent stem cells.

### (Configuration 24)

The information processing system according to Configuration 23, wherein the first information is information regarding a morphology of an outer peripheral portion of the colony of pluripotent stem cells.

### (Configuration 25)

The information processing system according to Configuration 24, wherein the information regarding the morphology of the outer peripheral portion of the colony of pluripotent stem cells includes at least one of information regarding spikes present in the outer peripheral portion of the colony of pluripotent stem cells or information regarding a cell present in the outer peripheral portion of the colony of pluripotent stem cells.

### (Configuration 26)

The information processing system according to Configuration 25, wherein the information regarding the spikes present in the outer peripheral portion of the colony of pluripotent stem cells is information regarding the number of spikes present in the outer peripheral portion of the colony of pluripotent stem cells.

### (Configuration 27)

The information processing system according to Configuration 25, wherein the information regarding the cell present in the outer peripheral portion of the colony of pluripotent stem cells is information regarding a cell present within a predetermined range from an outer periphery of the colony of pluripotent stem cells.

### (Configuration 28)

The information processing system according to Configuration 27, wherein the information regarding the cell presents within the predetermined range from the outer periphery of the colony of pluripotent stem cells is information regarding a nucleus of the cell present within the predetermined range from the outer periphery of the colony of pluripotent stem cells.

### (Configuration 29)

The information processing system according to Configuration 28, wherein the information regarding the nucleus of the cell present within the predetermined range from the outer periphery of the colony of pluripotent stem cells includes at least one of information regarding a size, information regarding an area, information regarding a circumferential length, information regarding a major axis length, or information regarding a minor axis length, of the nucleus of the cell present within the predetermined range from the outer periphery of the colony of pluripotent stem cells.

### (Configuration 30)

The information processing system according to any one of Configurations 23 to 30, wherein the second information includes at least one of information regarding a determination result of whether the pluripotent stem cells include chromosomally abnormal cells, information regarding a proportion of the chromosomally abnormal cells included in the pluripotent stem cells, or information indicating a type of a chromosomal abnormality included in the pluripotent stem cells.

### (Configuration 31)

The information processing system according to any one of Configurations 23 to 30, further including a first information generation unit configured to generate the first information by analyzing the image data including the colony of pluripotent stem cells acquired by the image data acquisition unit.

### (Configuration 32)

The information processing system according to any one of Configurations 23 to 31, wherein the second information generation unit is configured to generate the second information based on the first information and third information different from the first information acquired in advance.

### (Configuration 33)

The information processing system according to Configuration 32, wherein the third information includes at least one of the following: culture conditions for the pluripotent stem cells; a type of a line of the pluripotent stem cells; or information regarding a predetermined morphology of a colony of pluripotent stem cells serving as a comparison target which has been acquired from the image data including the colony of pluripotent stem cells serving as the comparison target.

### (Configuration 34)

The information processing system according to any one of Configurations 23 to 33, wherein the second information is information regarding a chromosome 12 abnormality in the pluripotent stem cells.

### (Configuration 35)

The information processing system according to any one of Configurations 23 to 34, further including a photographing unit configured to pick up an image including the colony.

### (Configuration 36)

The information processing system according to any one of Configurations 23 to 35, wherein the first information acquisition unit is configured to acquire the first information generated by inputting the image data including the colony of pluripotent stem cells to a first trained model obtained through training based on training data including the image data including the colony of pluripotent stem cells and a scalar value of the first information serving as a label corresponding to the image data.

### (Configuration 37)

The information processing system according to any one of Configurations 23 to 36, wherein the second information generation unit is configured to generate the second information by inputting the first information to a second trained model obtained through training based on training data including: a scalar value of the first information that is the information regarding the predetermined morphology of the colony of pluripotent stem cells; and types of chromosomal abnormalities of the colony of pluripotent stem cells serving as a label corresponding to the scalar value.

### (Configuration 38)

The information processing system according to any one of Configurations 23 to 36, wherein the second information generation unit is configured to refer to information regarding a correspondence relationship between the first information and the second information to generate the second information from the first information.

### (Configuration 39)

The information processing system according to any one of Configurations 23 to 38, wherein the second information generation unit is configured to generate the second information based on a predetermined threshold value that is based on the information regarding the predetermined morphology of the colony.

### (Configuration 40)

The information processing system according to any one of Configurations 23 to 39, further including a display control unit configured to perform control to display the second information on a display unit.

### (Configuration 41)

The information processing system according to Configuration 40, wherein the display control unit is configured to perform control to display the first information together with the second information on the display unit.

### (Configuration 42)

The information processing system according to any one of Configurations 23 to 41, wherein the display control unit is configured to perform control to display time-series data of the second information on a display unit.

### (Configuration 43)

The information processing system according to any one of Configurations 40 to 42, further including the display unit.

### (Method 1)

An information processing method including:
acquiring image data including a colony of pluripotent stem cells;
acquiring, based on analysis of the image data, first information regarding a predetermined morphology of the colony of pluripotent stem cells; and
generating, based on the first information, second information that is information regarding chromosomal abnormalities in the pluripotent stem cells.

### (Method 2)

The information processing method according to Method 1, further including analyzing the image data.

### (Method 3)

A non-transitory storage medium storing a program that causes a computer to execute each of the steps of the information processing method of Method 1 or 2.

### (Manufacturing Method 1)

A cell manufacturing method including:
acquiring image data including a colony of pluripotent stem cells;
acquiring, based on analysis of the image data, first information that is information regarding a predetermined morphology of the colony of pluripotent stem cells;
generating, based on the first information, second information that is information regarding chromosomal abnormalities in the pluripotent stem cells; and
classifying, based on the second information, pluripotent stem cells being cultured which include the pluripotent stem cells.

According to the present disclosure, the information regarding the chromosomal abnormalities in the pluripotent stem cells can be acquired non-invasively and efficiently.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

### OTHER EMBODIMENTS

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

This application claims the benefit of Japanese Patent Application No. 2024-143346, filed Aug. 23, 2024, and Japanese Patent Application No. 2025-137192, filed Aug. 20, 2025, which are hereby incorporated by reference herein in their entirety.

What is claimed is:

1. An information processing system comprising:
an image data acquisition unit configured to acquire image data including a colony of pluripotent stem cells;
a first information acquisition unit configured to acquire, based on analysis of the image data, first information that is information regarding a predetermined morphology of the colony of pluripotent stem cells; and
a second information generation unit configured to generate, based on the first information, second information that is information regarding chromosomal abnormalities in the pluripotent stem cells.

2. The information processing system according to claim 1, wherein the first information comprises information regarding a morphology of an outer peripheral portion of the colony of pluripotent stem cells.

3. The information processing system according to claim 2, wherein the information regarding the morphology of the outer peripheral portion of the colony of pluripotent stem cells includes at least one of information regarding spikes present in the outer peripheral portion of the colony of pluripotent stem cells or information regarding a cell present in the outer peripheral portion of the colony of pluripotent stem cells.

4. The information processing system according to claim 3, wherein the information regarding the spikes present in the outer peripheral portion of the colony of pluripotent stem cells comprises information regarding the number of spikes present in the outer peripheral portion of the colony of pluripotent stem cells.

5. The information processing system according to claim 1, wherein the second information includes at least one of information regarding a determination result of whether the pluripotent stem cells include chromosomally abnormal cells, information regarding a proportion of the chromosomally abnormal cells included in the pluripotent stem cells, or information indicating a type of a chromosomal abnormality included in the pluripotent stem cells.

6. The information processing system according to claim 1, further comprising a first information generation unit configured to generate the first information by analyzing the image data including the colony of pluripotent stem cells acquired by the image data acquisition unit.

7. The information processing system according to claim 1, wherein the second information generation unit is configured to generate the second information based on the first information and third information different from the first information acquired in advance.

8. The information processing system according to claim 7, wherein the third information includes at least one of the following: culture conditions for the pluripotent stem cells; a type of a line of the pluripotent stem cells; or information regarding a predetermined morphology of a colony of pluripotent stem cells serving as a comparison target which has been acquired from image data including the colony of pluripotent stem cells serving as the comparison target.

9. The information processing system according to claim 1, wherein the second information comprises information regarding a chromosome 12 abnormality in the pluripotent stem cells.

10. The information processing system according to claim 1, further comprising a photographing unit configured to pick up an image including the colony.

11. The information processing system according to claim 1, wherein the first information acquisition unit is configured to acquire the first information generated by inputting the image data including the colony of pluripotent stem cells to a first trained model obtained through training based on training data including the image data including the colony of pluripotent stem cells and a scalar value of the first information serving as a label corresponding to the image data.

12. The information processing system according to claim 1, wherein the second information generation unit is configured to generate the second information by inputting the first information to a second trained model obtained through training based on training data including: a scalar value of the first information that is the information regarding the predetermined morphology of the colony of pluripotent stem cells; and types of chromosomal abnormalities of the colony of pluripotent stem cells serving as a label corresponding to the scalar value.

13. The information processing system according to claim 1, wherein the second information generation unit is configured to refer to information regarding a correspondence relationship between the first information and the second information to generate the second information from the first information.

14. The information processing system according to claim 1, wherein the second information generation unit is configured to generate the second information based on a predetermined threshold value that is based on the information regarding the predetermined morphology of the colony.

15. An information processing method comprising:
acquiring image data including a colony of pluripotent stem cells;
acquiring, based on analysis of the image data, first information regarding a predetermined morphology of the colony of pluripotent stem cells; and
generating, based on the first information, second information that is information regarding chromosomal abnormalities in the pluripotent stem cells.

16. The information processing method according to claim 15, further comprising analyzing the image data.

17. A non-transitory storage medium storing a program that causes a computer to execute each of the steps of the information processing method of claim 15.

18. A cell manufacturing method comprising:
acquiring image data including a colony of pluripotent stem cells;
acquiring, based on analysis of the image data, first information that is information regarding a predetermined morphology of the colony of pluripotent stem cells;
generating, based on the first information, second information that is information regarding chromosomal abnormalities in the pluripotent stem cells; and
classifying, based on the second information, pluripotent stem cells being cultured which include the pluripotent stem cells.
